(19) United States
(12) Patent Application Publication
(10) Pub. No.: US 2024/0100239 A1
(43) Pub. Date: Mar. 28, 2024

(54) Probe System for Endoluminal Negative Pressure Therapy

(71) Applicant: ATMOS MedizinTechnik GmbH & Co. KG, Lenzkirch (DE)

(72) Inventors: Patrick ECKERT, Weilheim-Remetschwiel (DE); Theron WEINMANN, Uehlingen-Birkendorf (DE)

(73) Assignee: ATMOS MedizinTechnik GmbH & Co. KG, Lenzkirch (DE)

(21) Appl. No.: 18/357,735

(22) Filed: Jul. 24, 2023

(30) Foreign Application Priority Data
Jul. 25, 2022 (DE) 102022118531.1

(51) Int. Cl. A61M 1/00

(52) U.S. Cl. CPC A61M 1/90; A61M 1/84

(57) ABSTRACT

Some embodiments are directed to a probe system for endoluminal negative pressure therapy with a probe tube, and a probe body, connected with the probe tube, which includes a basic body with an inner lumen and with at least one opening which leads from the inner lumen through an outer wall of the basic body to an outer side of the basic body, in which the probe body includes on the outer side of the basic body several rod-form structures and/or at least one lamella-form structure.

### BACKGROUND

Negative pressure therapy, also referred to by the term vacuum therapy, is a form of treatment in which in the proximity of a chronic or acute wound or an inflamed site negative pressure is applied and, in most cases, continuously or periodically maintained or built-up over a relatively long time which, in particular, can contribute to improved wound healing, for example thereby that discharged wound secretions are effectively aspirated. An overview of application feasibilities of negative pressure therapy and negative pressure therapy devices can be found, for example, in DE 10 2016 114 819 A1.

However, it has been found in medical practice that such sponge bodies entail considerable risks leading to adhesions of the sponge body on the tissue forming the lumen wall. This limits the time during which a given probe body can remain in the lumen and establishes the necessity of multiple changes of the probe system for endoluminal negative pressure therapy over the healing course of an endoluminal wound which can extend over several weeks.

### SUMMARY

The application therefore addresses the problem of providing an improved probe system for endoluminal negative pressure therapy which can remain within the lumen over a long term. This problem is resolved through a probe system for endoluminal negative pressure therapy as disclosed herein. Advantageous further developments of some embodiments of the application are disclosed herein.

The probe system according to an embodiment for endoluminal negative pressure therapy comprises a probe tube and a probe body connected with the probe tube. The probe body has a basic body with an inner lumen and with at least one opening which originates at the inner lumen and leads through the outer wall of the basic body to an outer side of the basic body.

In many cases it is herein advantageous for the basic body to be cylindrical in form. The lumen can, for example, be formed by an opening or bore extending coaxially to the cylinder axis of the cylindrical body; the opening(s), which connect the inner lumen and the outer side of the basic body, can then preferably penetrate the outer wall, formed by the cylinder jacket, perpendicularly to the cylinder axis. It is herein preferred if several openings are provided which point in different directions such that during the generation of negative pressure a suction direction in multiple directions develops.

Essential to an embodiment is for the probe body to comprise on the outer side of the basic body, in particular on its outer jacket surface, several rod-form structures and/or at least one lamella-form structure. In some embodiments of the application these structures can herein also comprise a duct with an outlet port with the duct communicating with the inner lumen of the basic body.

Subsumed under the term “rod-form” are herein in particular structures whose length, measured from their starting point at the basic body up to their end opposite the basic body, is a multiple, preferably more than the fivefold, of the greatest extent of their cross section perpendicular to this longitudinal direction. The cross section can herein change along the longitudinal direction of the structure. Especially preferred are embodiments with a circular cross section such that rod-form structures can, in particular, have the geometry of a cylinder, of a truncated cone or of a cone whose height in each instance amounts to a multiple, preferably to more than the fivefold, of its greatest diameter.

Subsumed under the term “lamella-form” are herein in particular disks and tape-form structures if their height, measured from their starting point at the basic body up to their end opposite the basic body, amounts to a multiple, in particular preferably to more than the fivefold, of its thickness (defined in particular by the distance between their largest surfaces).

These rod-form and/or lamella-form structures ensure that the particular wall of the body opening or body cavity into which they are inserted is spaced apart from the basic body and cannot be aspirated due to the negative pressure into openings of the basic body of the probe body.

But even if several such structures are provided, these are simultaneously individual structures that are spaced apart such that, unlike as is the case with sponge-like structures of prior art, the risk of adhesions between probe body and tissue is drastically reduced.

The efficiency of removing wound secretions is herein surprisingly high although no absorption of the wound secretions into a sponge takes place, which can presumably be explained by the sponge representing a relatively high suction resistance, such that the aspiration becomes more efficient.

The probe body of the probe system according to an embodiment entails special advantages when this probe system is not only employed for negative pressure therapy but also for introducing flushing solutions or medications for cleaning the wound or for releasing obstructions. Through the combination of flushing and aspiration sufficient drainage of wound secretions is ensured. In the case of sponge systems this combination is not available whereby they quickly become clogged.

In the probe body of the probe system according to an embodiment the liquid can be allowed to flow directly out of the openings of the basic body.

Experience gained so far, furthermore, indicates that through the structures on the probe body according to an embodiment stimulation of the tissue promoting the healing process is effected.

It is especially preferred for the basic body and the probe tube to be implemented integrally or to be connected with one another under material closure. In this way the integrity of the probe system can also be especially safely ensured during the insertion into or the removal out of the lumen. For the same reason the integral implementation or material-closure connection between basic body and the structures is also preferred.

Especially simple to produce are structures that extend perpendicularly away from the basic body and/or are perpendicular to the outer side of the basic body, thus toward its outer wall through which lead the openings, when the probe body is not introduced into a body cavity.

If the structures are disposed annularly or helically on the outer wall of the basic body they ensure the simultaneous spacing of the basic body from the wall of the body opening, into which the probe body has been inserted, in all directions that are perpendicular to the course or extension of the basic body. This reduces the probability of openings becoming clogged and increases the probability of unclogged openings being available.

It is especially preferred for the structures to be flexible and/or elastic. There are in particular two reasons for this preference: firstly, they can in this case become deformed when negative pressure is applied and possibly even come into contact on the basic body, which improves the aspiration efficiency, however they can continue to ensure that the body cavity does not completely collapse onto the surface of the basic body since the body cavity is still kept at a distance from the basic body due to the structures that, for their part, are still in contact on the basic body. Secondly, the findings up to now appear to indicate that in this way an especially efficient stimulation of the tissue is attained which promotes wound healing.

Thereby that the length, thus the distance between their starting points on the basic body and the end opposite the basic body, of the structures is varied, the probe body can be adapted with respect to its geometry and can in this way be devised for example such that it is especially readily introducible into the body cavity.

An especially preferred embodiment of the application provides for the probe tube to be a biluminal tube. In addition to the negative pressure therapy function, this implementation allows in an especially simple manner realizing a flushing function in which a flushing solution, for example an NaCl solution, is supplied through one of the lumina and aspirated through the other lumen. A medically important advantage of biluminal systems is that secretion that has already been aspirated and is already in the aspiration lumen is not carried back into the body opening, for example an intestine, through the flushing process. Furthermore, thereby the flushing time with a biluminal system becomes shorter.

In the fundamentally likewise feasible use of a probe tube with only one lumen when switching between flushing operation and negative pressure operation it is necessary to ensure that the particular solution or vacuum source is completely sealed against the probe tube in order to ensure the correct execution of the action currently to be completed while in a biluminal probe tube such sealing does not need to be carried out anew with each switchover but only once when connecting the probe tube to the particular source.

It is herein preferred for the two lumina of the biluminal tube to have cross sectional areas of different magnitude and for the flushing solution to be supplied through the lumen with the smaller cross sectional area. Especially preferred is an embodiment in which the cross sectional area of the lumen utilized for aspiration is the largest feasible area. The cross section to which reference is made here is a section perpendicular to the running direction of the probe tube.

Such biluminal probe tube is especially simple of production if the cross sections of both lumina of the biluminal tube are circular in shape. Better optimization of the cross sectional area utilized for aspiration can be attained if the cross section of one of the lumina of the biluminal tube is circular and the cross section of the other lumen of the biluminal tube is crescent-shaped wherein the concave side of the crescent is oriented toward the circular lumen.

A still larger cross sectional area of the lumen utilized for aspiration can be attained if the two lumina of the biluminal tube, viewed in cross section, are separated from one another by a straight wall, such as is the case, for example, if the wall extends like a chord through a lumen with circular cross section and thereby separates it into two circle segment-shaped lumina.

If the flushing function as well as also the negative pressure therapy function are to be realized, it can be of advantage to provide also two lumina in the basic body of the probe which, in particular, also enables the simultaneous application of both functions. If the flushing as well as also the aspiration are to take place over the entirety of the basic body periphery, these can, for example, be devised as two encircling helical coils or a double helix: however, they can also simply extend parallel to one another. Accordingly, in this case the openings in the basic body have in this case either only flushing function or only aspiration function while, otherwise, their function would change depending on the particular active operating mode.

For the precise positioning of the probe head it is advantageous if on the outer side of the probe tube a marking is placed such that the position of the probe system in a lumen into which it is inserted can be read off.

Alternatively or additionally, a measure to be carried out for enabling the precise positioning of the probe body in the interior of the lumen comprises applying on the probe tube a radiopaque stripe and/or adding to the material of which the probe tube and/or the probe body is comprised a radiopaque agent. Determining the precise position of the probe body within the lumen based on an X-ray image is thereby enabled.

Thereby that the inner lumen of the basic body on the side opposite the connection site to the probe tube is closed with a plug, thereby generating an opening at the distal end of the basic body into wall sections of the body cavity, into which the probe body is introduced, or relatively large particles present therein, are aspirated into the inner lumen and/or clog it is avoided. This could in principle also be attained with a lumen closed in the distal direction by a wall which, however, in a length adaptation of the basic body leads to the problems described above. In this way the plug also contributes to enabling a length adaptation of the probe body.

If at the side opposite the connection site to the probe tube, thus at the distal side of the basic body, a loop is disposed, the probe can be readily introduced with the aid of endoscopic instruments.

It is especially advantageous for the probe body and the probe tube to be comprised of silicone. This material exhibits very good biocompatibility and high chemical resistance against various media. It is simultaneously soft and flexible and tear-resistant.

In the following the application will be described in greater detail in conjunction with Figures showing embodiment examples. Therein depict:

### BRIEF DESCRIPTION OF THE DRAWINGS

FIG. 1a a plan view onto a first embodiment example of a probe system;

FIG. 1b: a cross section through the probe system of FIG. 1a;

FIG. 2a: a plan view onto a probe body of a second embodiment example of a probe system;

FIG. 2b: an enlarged representation of the plug of the probe body of FIG. 2a;

FIG. 3a: a probe body of a third embodiment example of a probe system;

FIG. 3b: a probe body of a fourth embodiment of a probe system;

FIG. 3c: a probe body of a fifth embodiment example of a probe system;

FIG. 3d: a probe body of a sixth embodiment example of a probe system;

FIG. 3e: a probe body of a seventh embodiment example of a probe system;

FIG. 3f: a probe body of an eighth embodiment example of a probe system;

FIG. 3g: a probe body of a ninth embodiment example of a probe system;

FIG. 4a: a cross section through a first variant of a biluminal probe tube;

FIG. 4b: a cross section through a second variant of a biluminal probe tube; and

FIG. 4c: a cross section through a third variant of a biluminal probe tube.

### DETAILED DESCRIPTION

For differing representations of identical embodiments identical reference symbols are used. However, for maintaining greater clarity of the Figures not all reference symbols are always entered. In particular structures or components present in large number in an embodiment, for example the openings in the basic body and rod- or lamella-form structures, are only provided by example with one reference symbol assigned to them which is valid for all of these structures or components of a given embodiment.

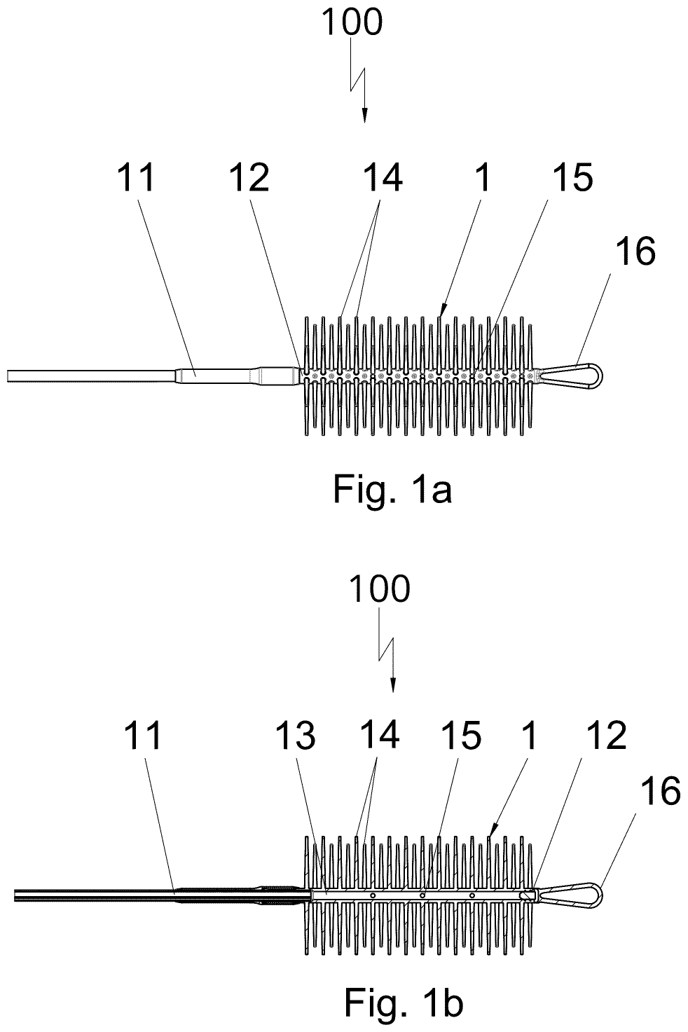

FIGS. 1a and 1b show two plan views of a first probe system 100. The probe system 100 comprises a probe tube 11 connected with the, in this case, cylindrical basic body 12 of a probe body 1. As FIG. 1b clarifies, the lumen of the probe tube 11 communicates with an inner lumen 13 of the basic body 12 extending coaxially to the cylinder axis of the cylindrical basic body 12, the axis defining its direction of extension, which basic body 12 at the distal end, opposite to the probe tube 11, is closed. On the outer side of basic body 12 a multiplicity of rod-form structures 14 are formed on which originate perpendicularly, thus in the radial direction with respect to the cylinder axis of the cylindrical base body 12, from the outside of basic body 12. The rod-form structures 14 are grouped into several groups, wherein the point of origin of the rod-form structures 14 of a given group are each located on the surface of the basic body 12 on a ring such that they are arranged annularly. Starting from the inner lumen 13 of the basic body 11 openings 15 lead to its outer side. Accordingly, across the lumen of the probe tube 11, the inner lumen 13, and the openings 15 liquid can be aspirated from the area surrounding the probe body 1 or be introduced into the area surrounding the probe body 1. At the distal end of the basic body 12, moreover, a loop 16 is disposed in order to facilitate placing the probe body 1.

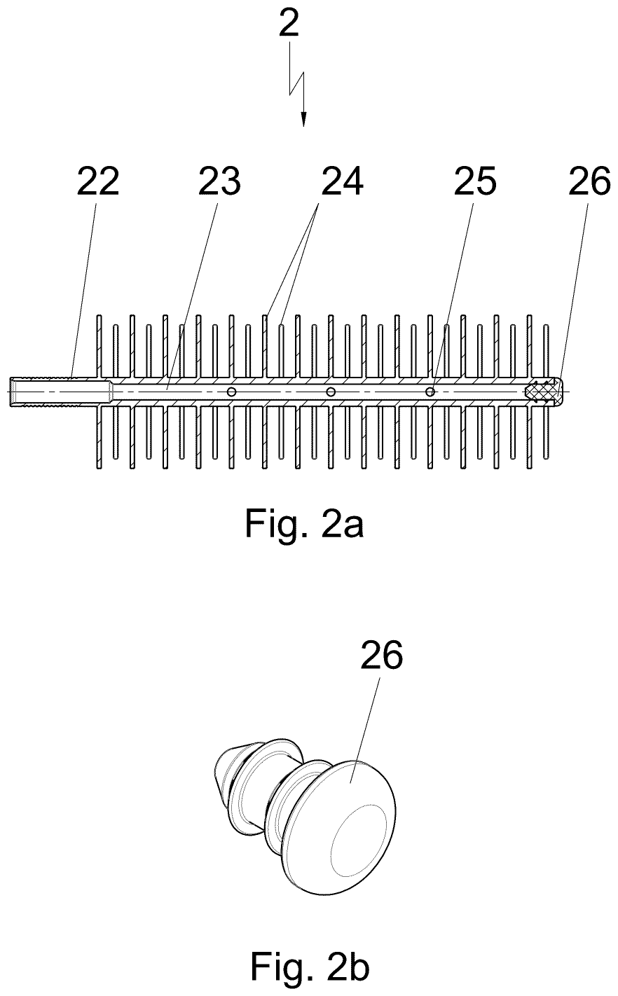

For the formation of a probe system 100 the probe bodies 2, 3, 4, 5, 6, 7, 8, 9 depicted in FIGS. 2a and 2b as well as 3a to 3g can each be connected with a probe tube in the manner shown in FIGS. 1a and 1b. The probe tube belonging to each particular probe system is therefore not depicted in these Figures.

The probe body 2 depicted in FIG. 2a with cylindrical basic body 22 with inner lumen 23 and rod-form structures 24 and openings 25 differs from the probe body 1 by its length and the number of rings of rod-form structures 24 disposed thereon. The probe body 2 can in fact be obtained by shortening probe body 1; the opening generated thereby at the distal end of probe body 2 is closed with the plug 26 shown in FIG. 2b in enlargement.

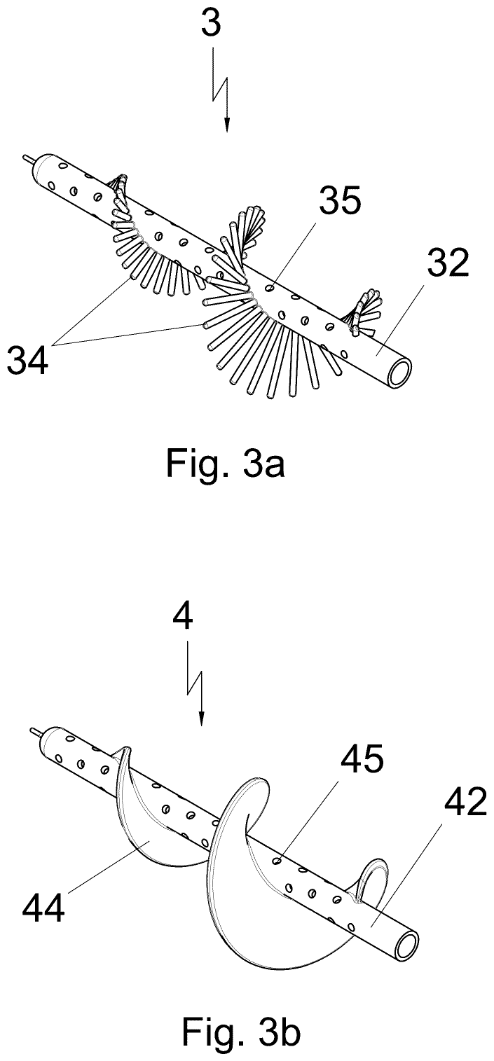

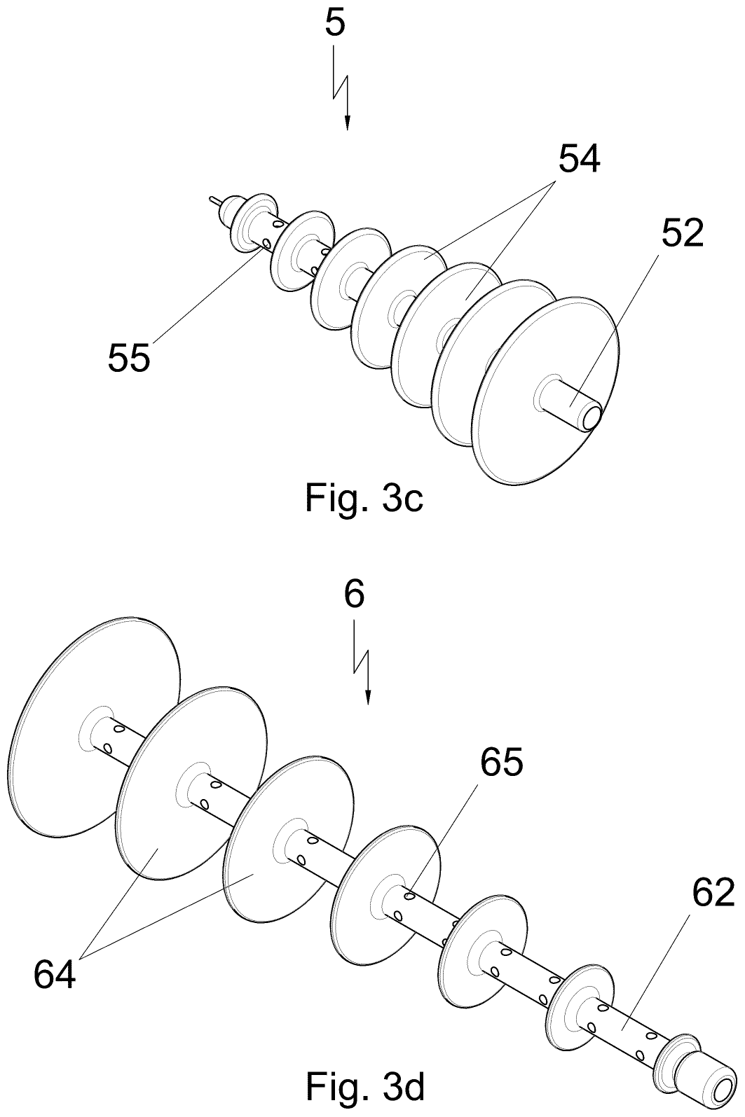

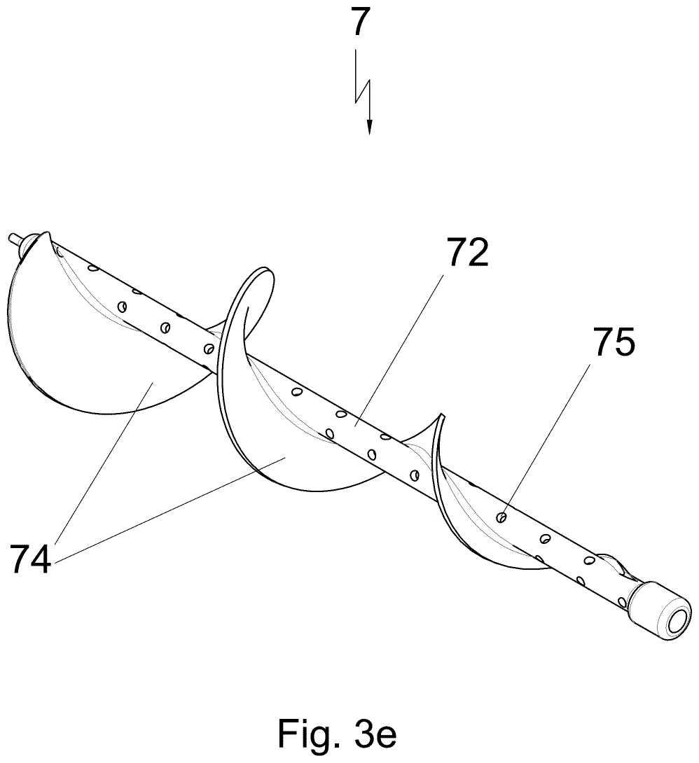

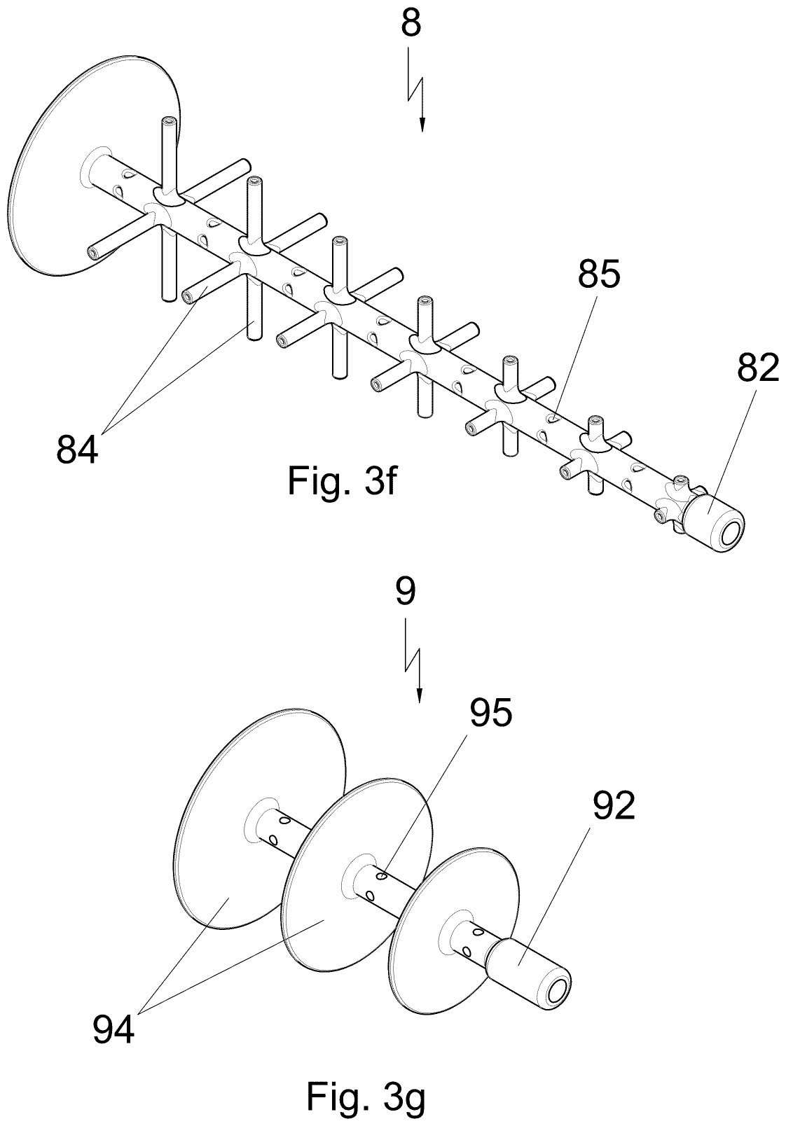

The probe bodies 3, 4, 5, 6, 7, 8, 9 shown in FIGS. 3a to 3g with cylindrical basic bodies 32, 42, 52, 62, 72, 82, 92 with inner lumen not depicted in the Figures and openings 35, 45, 55, 65, 75, 85, 95 differ with respect to form and distribution of the rod-form and/or lamella-form structures 34, 44, 54, 64, 74, 84 disposed thereon. The form and distribution preferred in each instance of the structures 34, 44, 54, 64, 74, 84 can herein in particular depend on anatomical conditions of the body cavity and/or on the wound to be treated.

In FIG. 3a rod-form structures 34 are disposed helically about the basic body 32, the length of which structures 34 initially increasing continuously from proximal to distal and subsequently continuously decreasing again.

In FIGS. 3b and 3e a lamella-form structure 44, respectively 74, is disposed in helical form coiled about the basic body 42 respectively 72, wherein the number of coils differs and the height of the coils in the case of probe body 4 increases from proximal to distal while in the case of probe body 7 it decreases from proximal to distal.

In each of FIGS. 3c, 3d and 3g several lamella-form structures 54, 64, 94 in the form of disks of differing diameters are formed thereon concentrically with respect to the cylinder axis of the cylindrical body 52, 62, 92. The diameter of the lamella-form structures 54 herein increases significantly in FIG. 3c from proximal to distal, in FIG. 3d the diameter of the lamella-form structures 64 decreases significantly from proximal to distal and in FIG. 3g the diameter of the lamella-form structures 94 decreases minimally from proximal to distal. In addition, the distance and the number of lamella-form structures 54, 64, respectively 94, are varied.

The probe body 8 depicted in FIG. 3f has at its proximal end a lamella-form structure 84, implemented as a disk, as well as a multiplicity of rod-form structures 84 of which in each instance four form a group disposed in the form of a cross at a given site of the basic body 82. The length of the rod-form structures 84 of each particular group herein decreases continuously from proximal to distal.

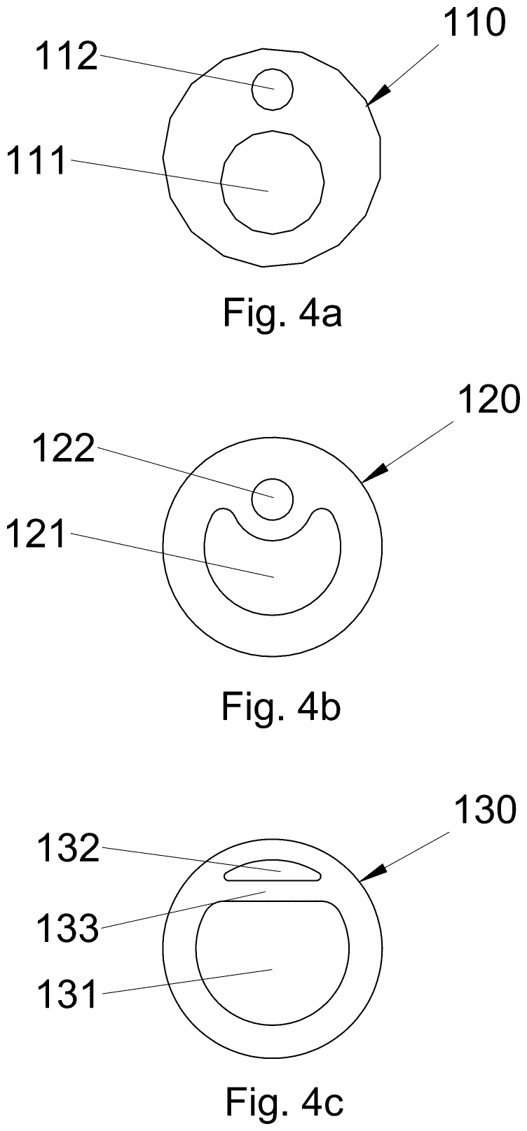

FIGS. 4a to 4c show three feasible cross sections of biluminal probe tubes 110, 120, 130 which can be utilized in order to realize in simple manner flushing and negative pressure operation of the probe system thereby that for each operating mode a separate lumen is provided. The probe tubes 110, 120, 130 accordingly are implemented biluminally with lumina 111, 112; 121, 122 respectively 131, 132, wherein lumina 112, 122, 132 intended for flushing operation have each a smaller cross section. While the embodiment according to FIG. 4a is especially simple of production, in the embodiment of FIG. 4c the cross section of lumen 131 is maximized which entails advantages for aspiration.

In FIG. 4a the cross section of both lumina 111, 112 of the biluminal tube is specifically circular while in FIG. 4b the cross section of one of the lumina 122 of the biluminal tube is circular and the cross section of the other lumen 121 of the biluminal tube is crescent-shaped, wherein the concave side of the crescent is oriented toward the circular lumen 122. In the embodiment of FIG. 4c the two lumina 131, 132 of the biluminal tube, viewed in cross section, are separated from one another by a straight wall 133 whereby an especially large volume can be generated for the purpose of aspiration.

### LIST OF REFERENCE NUMBERS

Probe body 1, 2, 3, 4, 5, 6, 7, 8, 9
Probe tube 11, 110, 120, 130
Basic body 12, 22, 32, 42, 52, 62, 72, 82, 92
Inner lumen 13, 23
Structure 14, 24, 34, 44, 54, 64, 74, 84, 94
Opening 15, 25, 35, 45, 55, 65, 75, 85, 95
Loop 16
Plug 26
Probe system 100
Lumen 111, 112, 121, 122, 131, 132
Wall 133

1. A probe system for endoluminal negative pressure therapy, comprising:
a probe tube;
a probe body, connected with the probe tube, the probe body comprising:
a basic body with a basic body outer wall and an outer side;
an inner lumen:
an opening which leads from the inner lumen through the basic body outer wall to the outer side of the basic body; and
a plurality of rod-form structures and/or a lamella-form structure on the outer side of the basic body.

2. The probe system as in claim 1, wherein the basic body and the probe tube are implemented integrally or are connected with one another under material closure.

3. The probe system as in claim 1, wherein the plurality of rod-form structures and/or the lamella-form structure extend perpendicularly to the basic body outer wall.

4. The probe system as in claim 1, wherein the rod-form structures and/or the lamella-form structure are disposed annularly or helically on the basic body outer wall.

5. The probe system as in claim 1, wherein the rod-form structures and/or the lamella-form structure are flexible and/or elastic.

6. The probe system as in claim 1, wherein a length of the rod-form structures and/or the lamella-form structure is varied.

7. The probe system as in claim 1, wherein the probe tube is a biluminal tube.

8. The probe system as in claim 7, wherein cross sections of the two lumina of the biluminal tube have different cross sectional areas.

9. The probe system as in claim 7, wherein cross sections of both lumina of the biluminal tube are circular.

10. The probe system as in claim 7, wherein a cross section of one of the lumina of the biluminal tube is circular and a cross section of the other lumina of the biluminal tube is crescent-shaped wherein the concave side of the crescent is oriented toward the circular lumen.

11. The probe system as in claim 7, wherein two lumina of the biluminal tube viewed in cross section are separated by a straight wall.

12. The probe system as in claim 1, further comprising a marking disposed on an outer side of the probe tube such that a position of the probe system in a lumen of a body cavity into which it is inserted can be read off.

13. The probe system as in claim 1, wherein a radiopaque stripe is applied on the probe tube and/or radiopaque agent is added to a material of the probe tube and/or the probe body.

14. The probe system as in claim 1, further comprising a plug that closes the inner lumen of the basic body on a side opposite the connection site to the probe tube.

15. The probe system as in claim 1, further comprising a loop on the side of the basic body opposite a connection site.

16. The probe system as in claim 1, wherein the probe body and the probe tube are comprised of silicone.
